(19) United States
(12) Patent Application Publication
(10) Pub. No.: US 2007/0049867 A1
(43) Pub. Date: Mar. 1, 2007

(54) System for treating chronic total occlusion caused by lower extremity arterial disease

(76) Inventor: Larry Earl Shindelman, Princeton, NJ (US)

Correspondence Address: MCCARTER & ENGLISH, LLP, FOUR GATEWAY CENTER, 100 MULBERRY STREET, NEWARK, NJ, 07102, US

(21) Appl. No.: 11/499,588

(22) Filed: Aug. 4, 2006

Related U.S. Application Data
(63) Continuation-in-part of application No. 11/197,968, filed on Aug. 5, 2005.

(51) Int. Cl. A61M 29/00

(52) U.S. Cl. 604/103.04; 606/192

(57) ABSTRACT

The present invention relates to a catheter system useful in treating lower extremity arterial chronic total occlusion (CTO). More particularly, the catheter system includes a first catheter having a first lumen extending therethrough, and a second catheter having a second lumen extending therethrough. The second catheter includes an engaging mechanism, such as an inflatable balloon, for engaging at least a portion of the first catheter such that a guide wire can be fed from the first lumen of the first catheter to the second lumen of the second catheter. In use, the first catheter is advanced to a treatment site through a vascular body from a downstream side of the treatment site. The second catheter is also advanced to the treatment site through the vascular body from an upstream side of the treatment site. The second catheter is engaged with the first catheter within the vascular body. The guide wire is then fed from the first catheter into the second catheter. Thereafter, the first and second catheters are removed from the vascular body, thereby leaving the guide wire extending through the treatment site. The guide wire is used to advance a treatment balloon to the treatment site for treating a CTO condition existing therein.

### CROSS-REFERENCE TO RELATED APPLICATION

The present application is a continuation-in-part of U.S. patent application Ser. No. 11/197,968 filed Aug. 5, 2005, the entire disclosure of which is incorporated herein by reference in its entirety.

### FIELD OF THE INVENTION

The present invention relates to dilation type balloon catheters, and diagnostic catheters for use in the treatment of stenotic regions within the arterial circulation. More particularly, the present invention relates to systems and methods for the treatment of chronic total occlusion (CTO) of the arterial circulation occurring in the lower extremities.

### BACKGROUND OF THE INVENTION

The arterial circulation is a system of tubes, comprised of a wall that defines a channel or lumen therein through which blood flows. In Peripheral Arterial Disease (PAD), the arterial wall becomes thickened and results in a corresponding reduction in the available area of the lumen through which blood flows. This reduction in the arterial lumen is called a stenosis. In the lower extremities, the thickening of the arterial wall is typically diffuse in nature, and can progress from a stenosis to a blockage or CTO of the arterial lumen. In addition to affecting the arteries of the lower extremities, PAD can affect all the arteries of the arterial system, leading to an increase risk of gangrene, heart attack, stroke and kidney disease.

One way to treat an arterial stenosis is with the use of a dilation balloon catheter, so as to widen the available area of the lumen through which blood flows. A guide-wire is placed percutaneously (through the skin), from a remote puncture site, into the lumen of the arterial system. Under X-ray control this guide-wire is negotiated through the arterial system, through areas of arterial thickening, and through the area of critical stenosis. The dilation balloon is tracked over this guide-wire to the area of critical arterial stenosis, whereupon inflation of the balloon with pressurized fluid, presses the inner area of arterial narrowing toward the outer wall of the blood vessel. The narrowed lumen now enlarges to the manufactured size of the balloon. The balloon dilation catheter is deflated and removed, leaving the available area of the arterial lumen enlarged to allow for the passage of an increased volume of blood.

The opportunity to treat lower extremity PAD is limited by the ability to gain successful guide-wire access through the area of arterial disease. In the treatment of a focal stenosis, guide-wire access is typically straightforward. In diffuse and complex arterial stenosis, however, guide-wire access is more difficult, and most problematic with chronic total occlusions (CTO).

In particular, in the case of CTO, the physician will insert a guide-wire into the arterial lumen, then pass that wire through the arterial lumen to the area of arterial disease. At the point of CTO, the physician will attempt to push the guide-wire through the occlusion by passing the wire from the arterial lumen proximal (upstream) to the occlusion, through the occlusion, and then returning the guide-wire to the arterial lumen distal (downstream) to the area of occlusion. In cases of CTO, when the guide-wire reaches the point of occlusion, it typically does not pass through the center of the occlusion, but “dissects” into the thickened arterial wall just proximal to the CTO. In this dissection plane, with the aid of a catheter, the guide-wire can traverse the area of the CTO. Once the guide-wire is distal to the area of CTO, while remaining within the dissection plane (within the thickened arterial wall) the physician attempts to return the leading edge of the guide-wire to the arterial lumen. With the leading edge of the guide-wire returned to the arterial lumen (distal to the CTO), the dilation balloon catheter is tracked over the wire, and positioned at the area of blockage. Once in place, the dilation balloon is inflated. Pushing outward against the occlusion, recanalization of the artery is established by the dilation balloon, with a luminal connection between the proximal arterial portion and the distal portion of the artery.

In the known systems, once the guide-wire traverses the CTO in the dissection plane, there is great difficulty and complexity involved in returning the guide-wire to the arterial lumen distal to the CTO. This difficulty often leads to failure to gain distal arterial luminal position of the wire, resulting in failure to successfully recanalize the area of CTO, leaving open surgical revascularization as the only alternative treatment option.

### SUMMARY OF THE INVENTION

The shortcomings and disadvantages of the prior art discussed above are overcome by providing an improved catheter system for positioning a guide wire through a treatment site within a vascular body. More particularly, the catheter system includes a first catheter having a first lumen extending therethrough, and a second catheter having a second lumen extending therethrough. The second catheter includes engaging means (e.g., at least one inflatable balloon) for engaging at least a portion of the first catheter such that a guide wire can be fed from the first lumen of the first catheter to the second lumen of the second catheter.

In use, the first catheter is advanced to a treatment site through a vascular body from a downstream side of the treatment site. The second catheter is also advanced to the treatment site through the vascular body from an upstream side of the treatment site. The second catheter is engaged with the first catheter within the vascular body adjacent the treatment site. A guide wire is then fed from the first catheter into the second catheter. Thereafter, the first and second catheters are removed from the vascular body, thereby leaving the guide wire extending through the treatment site. The guide wire is used to advance a treatment balloon to the treatment site for treating a CTO condition existing therein.

### BRIEF DESCRIPTION OF THE DRAWINGS

For a more complete understanding of the present invention, reference is made to the following detailed description of exemplary embodiments, considered in conjunction with the accompanying drawings, in which:

FIG. 1 is a perspective schematic illustration of a system for facilitating proper positioning of a capture balloon and associated guide-wires to facilitate treatment of a CTO within vascular bodies in accordance with a first exemplary embodiment of the present invention, the system including a balloon assembly, an angled catheter, and plural guide-wires;

FIG. 2 is a side cross-sectional view of the balloon assembly and the angled catheter of the system illustrated in FIG. 1;

FIG. 3 is a side elevational view of the balloon assembly of FIGS. 1 and 2 that shows certain radio-opaque markers used for alignment purposes;

FIG. 4 is a longitudinal cross-sectional view of an occluded region of a vessel showing the system of FIG. 1, except that the balloon assembly is uninflated and the angled catheter has been replaced by a straight catheter;

FIG. 5 is a cross-sectional view similar to that of FIG. 4, except that the straight catheter has been replaced by the angled catheter of FIGS. 1 and 2;

FIG. 6 is a schematic representation of how the apparatus of FIG. 5 would appear to a practitioner utilizing a radioscope display to confirm proper orientation and positioning of the angled catheter and the uninflated balloon assembly relative to each other;

FIG. 7 is a cross-sectional view similar to that of FIG. 5, except that the balloon assembly has now been inflated, causing the complete docking of the angled catheter and the balloon assembly;

FIG. 8 is a schematic representation of how the apparatus of FIG. 7 would appear to a practitioner utilizing a radioscope display to confirm proper coupling of the angled catheter and the now-inflated balloon assembly;

FIG. 9 is an enlarged-scale cross-sectional view of the completely docked angled catheter and balloon assembly of FIG. 7, a guide-wire being shown within the catheter;

FIG. 10 is a cross-sectional view similar to FIG. 9, except that the guide-wire has been advanced through the angled catheter and into the balloon assembly;

FIG. 11 is a cross-sectional view similar to FIG. 10, except that the angled catheter has not been completely docked with the balloon assembly;

FIG. 12 is a cross-sectional view similar to FIG. 5, showing the balloon assembly in a deflated state and the captured guide-wire advancing further upstream through the balloon assembly;

FIG. 13 is a perspective schematic illustration of a system constructed in accordance with a second exemplary embodiment of the present invention, the system including a capture catheter, which has head and tail balloons, an angled catheter, and plural guide-wires;

FIG. 14A is a side cross-sectional view of the capture catheter and the angled catheter of the system illustrated in FIG. 13;

FIG. 14B is a cross-sectional view, taken along section line 14B-14B and looking in the direction of the arrows, of the capture catheter shown in FIG. 14A;

FIG. 15 is a side elevational view of a portion of the capture catheter shown in FIGS. 13 and 14A, showing certain radio-opaque markers used for alignment purposes;

FIG. 16 is a side cross-sectional view of an occluded region of a blood vessel, the angled catheter and the capture catheter illustrated in FIG. 13 being deployed at the occluded region, the head and tail balloons being in their deflated states;

FIG. 17 is a schematic representation of how the system illustrated in FIG. 16 would appear to a practitioner utilizing a radioscope display;

FIG. 18 is a view similar to that of FIG. 16, except that the head and tail balloons are in their inflated states;

FIG. 19 is a schematic representation of how the system illustrated in FIG. 18 would appear to a practitioner utilizing a radioscope display;

FIG. 20 is an enlarged cross-sectional view of the angled catheter and the capture catheter which are properly engaged so to permit feeding of a guide-wire from the angled catheter to the capture catheter;

FIG. 21 is a cross-sectional view of a vascular vessel, taken along a plane substantially perpendicular to the longitudinal axis of the vascular vessel, a system constructed in accordance with a third embodiment of the present invention being shown schematically in FIG. 21; and

FIG. 22 is a cross-sectional view of a vascular vessel, taken along a plane substantially perpendicular to the longitudinal axis of the vascular vessel, a system constructed in accordance with a third embodiment of the present invention being shown schematically in FIG. 22.

### DETAILED DESCRIPTION OF THE INVENTION

For the purposes of the discussion below, “proximal” is defined as closer to the heart. Conversely, “distal” is defined as further from the heart. Additionally, the “downstream” direction in an artery is defined as the ordinary direction of blood flow (i.e., away from the heart) within the artery, whereas the “upstream” direction in an artery is defined as being opposite the “downstream” direction therein (i.e., toward the heart).

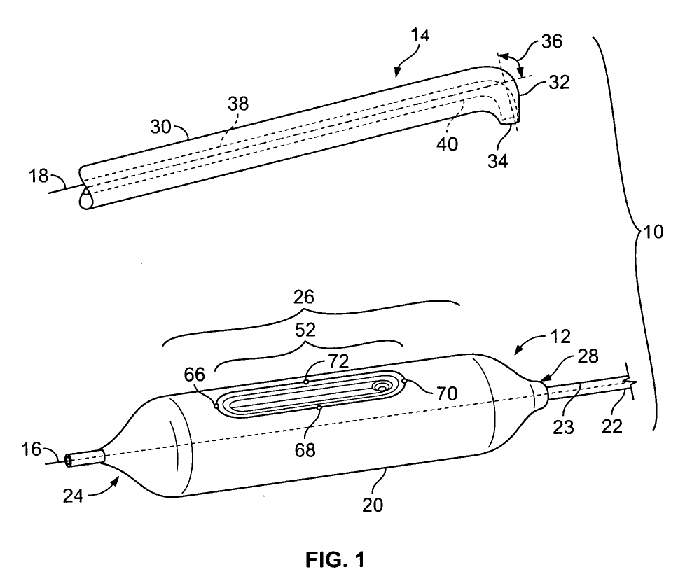

FIG. 1 is a perspective view of a system 10 for treating patients suffering from chronic total occlusion (hereinafter “CTO”) occurring in the lower extremities, in accordance with a first embodiment of the present invention. The system 10, which may be used in conjunction with the inventive methods described hereinbelow, includes a balloon assembly (also referred to herein as “capture catheter”) 12, an angled catheter 14, and first and second guide-wires 16, 18, respectively, both of which are of conventional construction. For purposes of clarity, the angled catheter 14 is shown in a scale somewhat larger than that of the balloon assembly 12.

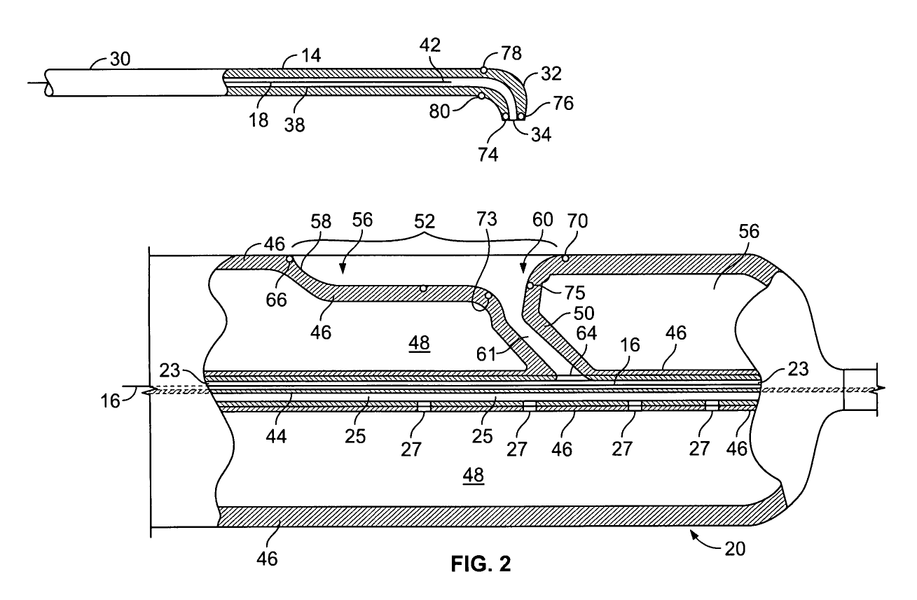

The balloon assembly 12 includes a balloon 20 (shown in a cigar-shaped inflated state), and an elongate tubular body 22 (i.e., a carrier). The balloon 20, which may also be referred to herein as a “capture balloon”, has a first end 24, a generally cylindrical middle portion 26, and a second end 28, and is attached to the elongate body 22 at both the first end 24 and the second end 28. The elongate body 22 is a flexible structure of conventional construction that is used to deliver/retrieve the balloon 20, and to permit the balloon 20 to be remotely inflated and deflated. For such purposes, the elongate body 22 is equipped with an axial lumen 23 (see FIG. 2) sized to accommodate the first guide wire 16, and a wall 44 used to create a separate internally-disposed passage 25 that is hydraulically coupled to the balloon 20 so as to permit a conventional inflation fluid to be delivered to and/or drained from the balloon 20 via holes 27 which are formed therein.

The angled catheter 14 (see FIG. 1) is of a construction similar in many respects to that of a straight catheter, but with some differences. For example, the angled catheter 14 includes an elongate portion 30 and a tapered end portion 32 (the latter terminating at a tip 34 of relatively small diameter), but the tapered end portion 32 is disposed at an angle 36 to the elongate portion 30, rather than being axially aligned therewith. Also, the tapered end portion 32 of the angled catheter 14 is conical at the tip 34, rather than rounded. Further, the angled catheter 14 includes a lumen 38 (see FIG. 1) which is sized to accommodate the second guide-wire 18. More particularly, the lumen 38 extends through the elongate portion 30 and the tapered end portion 32 and terminates at an opening which is formed in the tip 34 and which faces downwardly.

Referring now to FIGS. 1 and 2, the balloon 20 includes certain structures and other features enabling a competent practitioner to cause the balloon 20 to receive the tapered end portion 32 of the angled catheter 14 within a vascular body (e.g., a blood vessel), and to further receive or “capture” an end 42 of the second guide-wire 18. The balloon assembly 12 is further configured, particularly when used in a manner and for purposes to be described more fully hereinafter, to guide the end 42 of the second guide-wire 18 in a smooth and convenient fashion through the balloon 20, and into and through the lumen 23 of the elongate body 22. In this regard, the balloon 20 includes exterior walls 46, which can be considered generally to define an inflatable interior region 48 of the balloon 20. The balloon 20 also includes channel walls 50, as well as a trough 52 which opens up to the exterior surface of the balloon 20 for receiving the tapered end portion 32 of the angled catheter 14. More particularly, the trough 52, which is defined by the exterior walls 46 and/or the channel walls 50 of the balloon 20, is formed in the balloon middle portion 26 along a border or outer perimeter of the balloon 20.

The trough 52 of the balloon 20 features a capture zone 56 adjacent to the outer perimeter of the balloon 20, which includes a scalloped region 58. The scalloped region 58 is formed from the exterior walls 46 of the balloon 20 and is generally concave, relatively shallow, and elongated axially. The scalloped region 58 has a depth that is preferably at least as deep as the length of the tip 34 of the angle catheter 14 (which is preferably about 2 mm, but may be varied according to need). A funnel-shaped opening 60 is also formed from the channel walls 50 and extends inwardly in a generally radial direction from the trough 52 to the elongate body 20. More particularly, the funnel-shaped opening 60 includes a channel 61 (see FIG. 2) which is in a slanted orientation.

Now referring to FIG. 2, the axial lumen 23, which extends through the elongate body 22, is sized to accommodate the first guide-wire 16. As can be seen in FIG. 2, the funnel-shaped opening 60 is oriented relative to the axial lumen 23 at an angle less than 90° so as to facilitate passage of a guide-wire from the funnel-shaped opening 60 into the axial lumen 23. In this regard, the funnel-shaped opening 60 communicates with the axial lumen 23 through an aperture or orifice 64 formed in an tubular wall of the elongate body 22.

With reference to FIGS. 1 and 2, the balloon 20 and the angled catheter 14 are each equipped with small, discrete portions of radio-opaque material that are embedded at selected locations in the structural material of each such component. More particularly, the balloon 20 includes small radio-opaque portions in the form of markers 66, 68, 70, 72, which are arranged in spaced relation around the outer perimeter of the trough 52, and markers 73, 75, which are arranged around an entry section of the funnel-shaped opening 60. Also, the angled catheter 14 includes small, discrete radio-opaque portions in the form of markers 74, 76 disposed on opposite longitudinal sides of the tip 34, and markers 78, 80 disposed on opposite vertical sides of the elongate portion 30 adjacent the angle 32. The significance of the number and arrangement of these radio-opaque markers will be described in detail hereinafter.

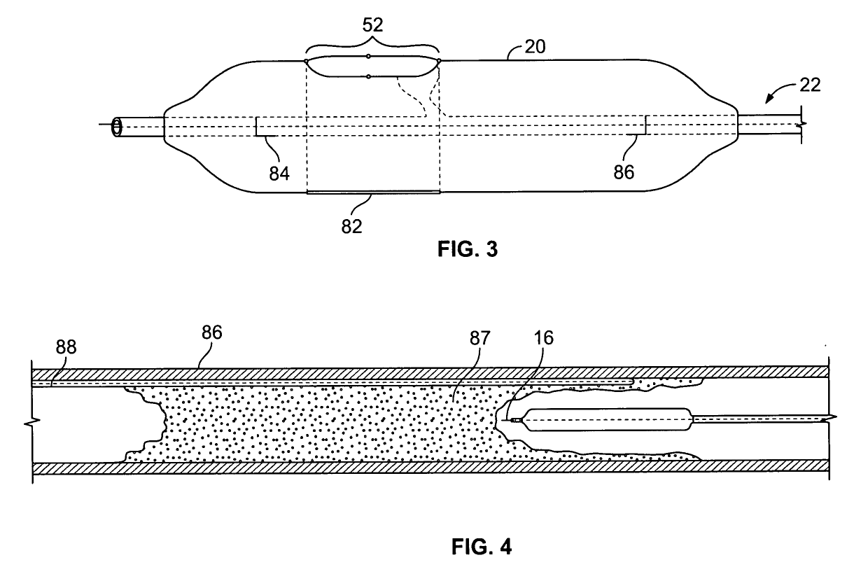

FIG. 3 shows that a lower portion 82 of the balloon 20 is coated and/or constructed of a radio-opaque material. The elongate body 22 also has a plurality of radio-opaque markers 84, 86, each of which has an L-shape and each of which is positioned on a side surface of the elongate body 22 to facilitate alignment of the trough 52 with the tip 34 of the angled catheter 14, as will be explained in greater detail hereinbelow.

As described below with reference to FIGS. 4 to 12, in operation, a competent practitioner can use the system 10 of FIGS. 1 to 3 to improve the axial positioning of the second guide-wire 18 within a totally occluded region (i.e., a treatment site) of a blood vessel. As described above, good axial positioning of a guide-wire improves the chances that a later-placed treatment balloon (not shown) will, when inflated, compress the blockage against the vessel wall in approximately equal amounts.

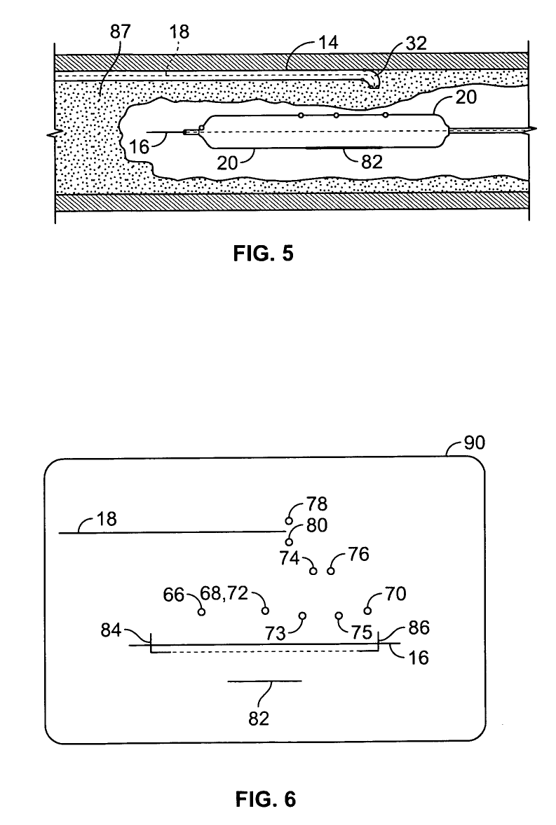

Referring to FIGS. 4 and 5, the first guide-wire 16, placed percutaneously, is advanced downstream through a vascular body or structure 86 (e.g., an arterial lumen) to a treatment site 87 (referred to hereinafter as “the CTO region”) where a CTO is present. If the CTO region 87 is present in a lower extremity of a patient, the first guide-wire 16 is preferably introduced into the vascular structure 86 through a puncture made at the patient's thigh portion. Once the first guide-wire 16 is properly positioned, the balloon 20 is then advanced along the first guide wire 16 until it is positioned adjacent the CTO region 87 (see FIG. 4). A second guide-wire 18 is also introduced into the vascular structure 86 from an area distal to the CTO region 87 (e.g., from an incision made in the patient's ankle or foot portion if the CTO region 87 is in a lower extremity of the patient). The second guide-wire 18 is advanced upstream to the CTO region 87 to a point just distal thereto. A conventional straight catheter 88, used in conjunction with the second guide-wire 18, is advanced upstream through the CTO region 87, in the plane of dissection (see FIG. 4). The catheter 88 facilitates the passage of the second guide-wire 18 through the plane of dissection, as it crosses the CTO region 87.

As shown in FIG. 5, the straight catheter 88 has been replaced by the angled catheter 14 along the second guide-wire 18. More particularly, the straight catheter 88 is withdrawn from the CTO region 87 by being pulled along the second guide-wire 18 and exiting through the skin of the patient at its original point of entry, leaving just the second guide-wire 18 in place within the vascular structure 86. The angled catheter 14 is then introduced to the patient via the point of entry used by the straight catheter 88, and advanced over the second guide-wire 18. The tapered end portion 32 of the angled catheter 14 is preferably made from an elastic material such that the tapered end portion 32 can be oriented from its normal, angled orientation (as shown in FIG. 2) to a substantially linear orientation relative to the elongate portion 30. As a result, the tapered end portion 32 can be passed through the CTO region 87 in its linear orientation so as to facilitate passage therethrough. Also, it is preferred that the tapered end portion 32 of the angled catheter 14 and the funnel-shaped opening 60 (FIG. 2) of the balloon 20 are nearly complementary in shape (for example, conical shape) such that the tapered end portion 32 can be “popped” into the funnel-shaped opening 60 upon inflation of the balloon 20. However, it should be understood by persons of ordinary skill in the art that the complementary shape is merely a preference, and is not required for proper operation of the invention.

After positioning the balloon 20 and the tapered end portion 32 of the angled catheter 14 at the CTO region 87, the axial and angular orientation of the balloon 20 and/or the tapered end portion 32 of the angled catheter 14 is adjusted for proper alignment/positioning. Referring to FIGS. 3 and 6, in order to properly position the balloon 20 relative to the angled catheter 14, a practitioner can use a radioscope display 90 (see FIG. 6) to remotely view the guide-wires 16 and 18, as well as the radio-opaque markers 66, 68, 70, 72, 73, 75, 82, 84, 86 (see FIGS. 1-3) of the balloon 20 and the radio-opaque markers 74, 76, 78, 80 (see FIG. 2) of the angled catheter 14. With the aid of the radioscope display 90, the balloon 20 and/or the angled catheter 14 can be moved axially and/or rotated relative to each other and/or around their respective guide-wires as necessary. For instance, images of the radio-opaque markers 84, 86 of the elongate body 22 appearing on the radioscope display 90 are used for adjusting the angular orientation of the balloon 20. More particularly, the balloon 20 is rotated until the vertical portions of the “L” shaped markers 84, 86 appear at their maximum on the radioscope display 90. Because the markers 84, 86 are arranged on a lateral surface of the elongate body 22, if the trough 52 of the balloon 20 is not in substantial angular alignment with the angled catheter 14, one or both of the markers 84, 86 may not be visible on the radioscope display 90, or their vertical portions may appear short. In order to adjust the angular orientation of the balloon 20, the balloon 20 is rotated until the marker 84, 86 become visible on the radioscope display 90 and/or until the respective vertical portions of the markers 84, 86 appear with their maximum lengths on the radioscope display 90. The angular orientation of the angled catheter can be adjusted in a similar manner by viewing the radio-opaque markers 74, 76 and/or the radio-opaque makers 78, 80.

One or more of the images appearing on the radioscope display 90 of the radio-opaque markers 66, 68, 70, 72, 73, 74, 75, 76, 78, 80 can also be used to verify whether the trough 52 and/or the funnel-shaped opening 60 are axially aligned with the tapered end portion 32 of the angled catheter 14. For instance, if the radio-opaque markers 74, 76 of the angled catheter 14 appear on the radioscope display 90 as being located axially between the radio-opaque markers 66, 70 of the balloon 20, such positioning indicates that the tapered end portion 32 is axially aligned with the trough 52. If such alignment is not indicated by the radioscope display 90, the angled catheter 14 and/or the balloon 20 can be moved axially to achieve proper alignment.

By the end of the alignment procedure discussed above, the tapered end portion 32 of the angled catheter 14 should be pointing directly toward the funnel-shaped opening 60 (FIG. 2) of the balloon 20, and vice versa. In this manner, when the balloon 20 is inflated, the tapered end portion 32 of the angled catheter 14 can properly engage the funnel-shaped opening 60, as will be discussed in greater detail hereinbelow.

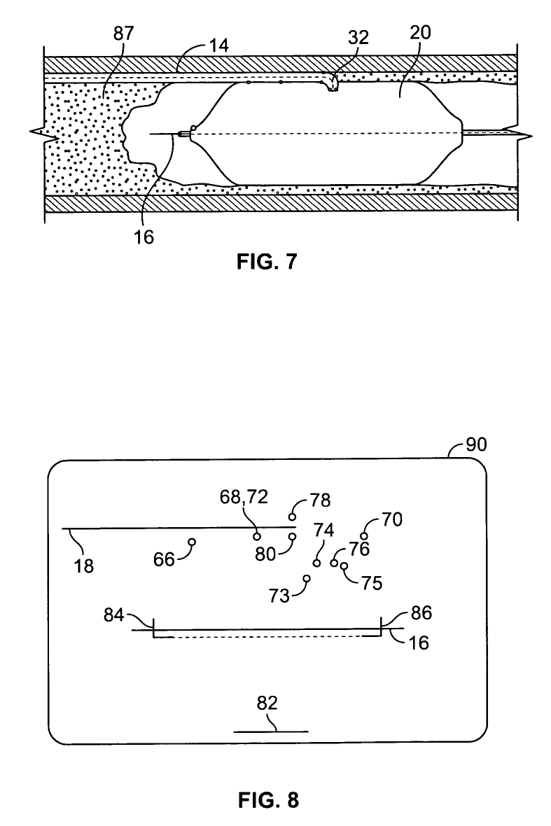

Referring now to FIG. 7, once proper alignment between the tapered end portion 32 of the angled catheter 14 and the funnel-shaped opening 60 (FIG. 2) of the balloon 20 has been achieved, the balloon 20 is inflated. Such inflation of the balloon 20 gives form to the trough 52 (FIG. 2) of the balloon 20, and eventually causes reactive forces from the walls of the balloon 20 to force the trough 52 and the tapered end portion 32 of the catheter 14 towards each other until the latter “pops” into the funnel-shaped opening 60 of the balloon 20. To the extent a small amount of axial or angular misalignment exists between the tapered end portion 32 and the trough 52 (FIG. 2) of the balloon 20 during or after inflation of the balloon 20, the tip 34 (FIG. 2) of the tapered end portion 32 can be caused to slide longitudinally or laterally along the surface of the scalloped region 58 (FIG. 2) as necessary to mate the parts. The practitioner can use the radioscope display 90 to remotely view (see FIG. 8) the guide-wires 16 and 18, and the radio-opaque markers 66, 68, 70, 73, 74, 75, 76, 78, 80, 82, 84, 86 of the balloon 20 and the angled catheter 14, so as to confirm proper mating has occurred between the angled catheter 14 and the balloon 20.

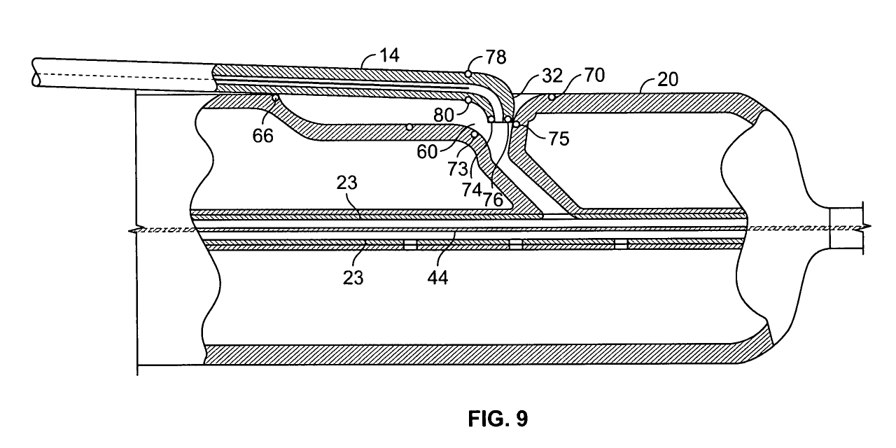

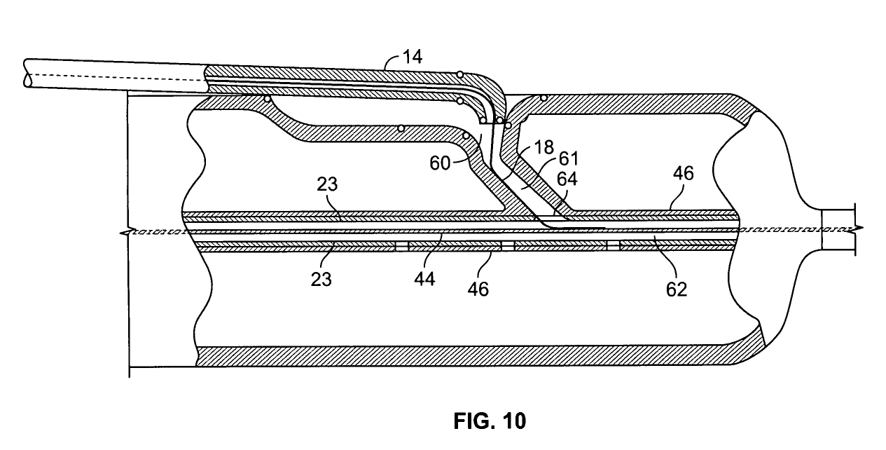

The nature of the mating relationship between the angled catheter 14 and the balloon 20 is illustrated in detail in FIG. 9. More particularly, the full and complete insertion of the tapered end portion 32 of the angled catheter 14 into the funnel-shaped opening 60 of the balloon 20, remotely confirmed by the practitioner via images appearing on the radioscope display 90, is shown in FIG. 9. (Note the similar comparative positions, as between FIGS. 6 and 8, of the guide-wires 16, 18, and the radio-opaque markers 74, 76, 78, 80, 84, 86 of the balloon 20 and the angled catheter 14.). The second guide wire 18 can now be advanced into the balloon 20, and into the lumen 23 (FIG. 2) and out of the vascular structure 86 of the patient. This process and a variation thereof will now be described below with reference to FIGS. 10-12.

As shown in FIG. 10, the first guide-wire 16 is removed from the lumen 23. This removal of the first guide-wire 16 allows for the advancement the second guide-wire 18 down the funnel-shaped opening 60 through the aperture 64 of the elongate body 22 into the lumen 23. Edges of the funnel-shaped opening 60 are adapted to permit the second guide-wire 18 to be snaked through the funnel-shaped opening 60 and into the axially disposed lumen 23 to thereby reduce the chances of the second guide-wire 18 accidentally bending in a wrong direction. Due to its slanted configuration, the channel 61 facilitates the passage of the guide-wire 18 therethrough and into the lumen 23.

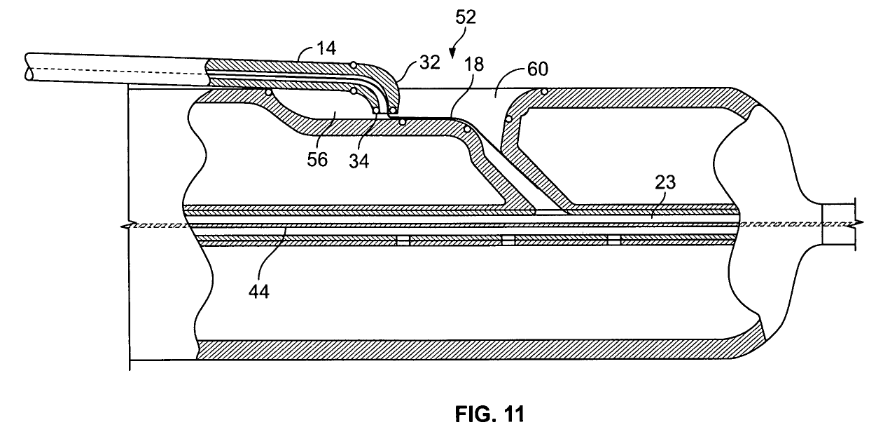

FIG. 11 illustrates a variation in the preferred alignment of the tapered end portion 32 of the angled catheter 14 with the funnel-shaped opening 60. In this particular case, the angled catheter 14 is arranged within the capture zone 56 of the trough 52, but the tapered end portion 32 of the angled catheter 14 is not in precise angular and/or axial alignment with the funnel-shaped opening 60 prior to inflation of the balloon 20, so the tapered end portion 32 has not “popped” into place in the funnel-shaped opening 60. Nevertheless, the practitioner can still advance the second guide-wire 18 into the funnel-shaped opening 60 and into the lumen 23. It is for this reason that the length of the tip 34 of the angled catheter 14 is preferably comparable to the depth of the trough 52, including but not limited to, for example, 2 mm to 2.5 mm. Also, the second guide-wire 18 should be flexible so that it can be advanced into the funnel-shaped opening 60 even though the tip 34 of the angled catheter 14 is not in its preferable position (aligned with the funnel-shaped opening 60).

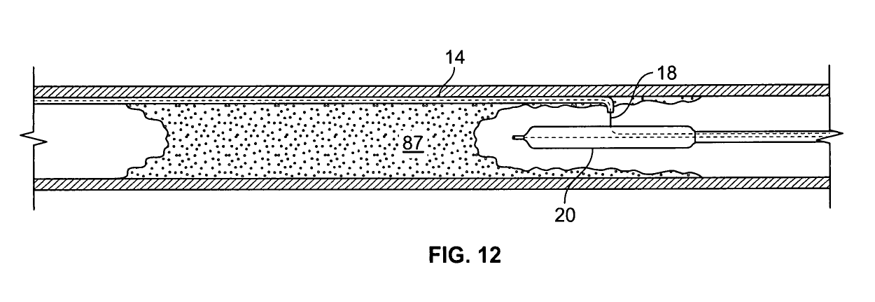

As shown in FIG. 12, the balloon 20, which has now captured the second guide-wire 18 such that the balloon 20 can now be deflated and decoupled from the angled catheter 14, is shown having returned to its uninflated state. The second guide-wire 18, a section of which is now directly visible in the space between the now-decoupled components, is advanced further upstream out of the balloon 20, through the elongate body 22 of the balloon assembly 12, and out of the vascular structure 86 of the patient such that the end 42 (see FIG. 2) of the second guide-wire 18 is outside of the patient's body and can be grasped or otherwise manipulated by the practitioner. At this point, the practitioner has a much greater ability to manipulate the axial position of the second guide-wire 18 (from two ends) than was the case when the end of the second guide-wire 18 was merely suspended in space at the upstream end of the CTO region 87 (see FIG. 4). The balloon 20 can now be removed from the vascular structure 86.

Now, although not shown, the second-guide wire 18 enters the body at a first entry point downstream of the CTO region 87 (e.g., at a foot or ankle region for treatment of a CTO in a lower extremity) and exits the body upstream of the CTO region 87 where the first guide-wire 16 entered through the skin (e.g., a thigh region for treatment of a CTO in a lower extremity). A conventional treatment balloon (not shown) can be tracked over the second guide-wire 18 from either the upstream or downstream entry points in the body (not shown). After positioning the treatment balloon in the desired location within the CTO region 87, the inflation of the treatment balloon pushes the CTO against the walls of the vascular structure 86, thus enlarging the opening made by the second guide-wire 18.

It should be noted that numerous advantages are provided by the system 10 of the present invention, and the above-described use of same to better position a treatment guide-wire relative to the axis of a vascular structure having a chronic total occlusion. For example, the number and locations of the radio-opaque markers present in the angled catheter 14 and the balloon 20 are advantageously selected and implemented so as to simplify, to the maximum extent possible, the task of the practitioner in rotating and moving the angled catheter 14 and the balloon 20 relative to each other as needed prior to coupling, and to verify proper coupling after inflation of the balloon 20. However, these markers can be rearranged, removed, or in certain cases, more markers can be added according to need. Also, the right-angle design embodied by the tapered end portion 44 of the angled catheter 14 and the funnel-shaped opening 60 of the balloon 20 reduces the actual coupling process to a simple “pop-in” step, according to which the practitioner need only inflate the balloon 20 toward the angled catheter 14, while simultaneously monitoring the radioscope display 90 to confirm a preferred method of coupling. Additionally, the present invention is configured to accommodate an imprecise arrangement where the tapered end portion 32 of the angled catheter 14 is positioned within the capture zone 56 of the trough 52 but not necessarily within the funnel-shaped opening 60, by allowing a practitioner to track the wire along the capture zone 56 and into the funnel-shaped opening 60. This variation in the method greatly simplifies and maximizes the chances of success in the subsequent balloon inflation/coupling step.

The system and method discussed above are particularly suitable for treating a CTO condition in a lower extremity, but the invention can be used for other vascular structures. For instance, typically, with regard to the present invention, a 4 French arterial sheath, which is known in the art (but not shown), can be placed within the lumen of the artery distal (away from the heart) to the CTO. In the lower extremity this artery is either the Posterior Tibial or Anterior Tibial Artery at the foot or ankle level. Under standard techniques the wire is advanced in a retrograde manner (going upstream) until the CTO is reached.

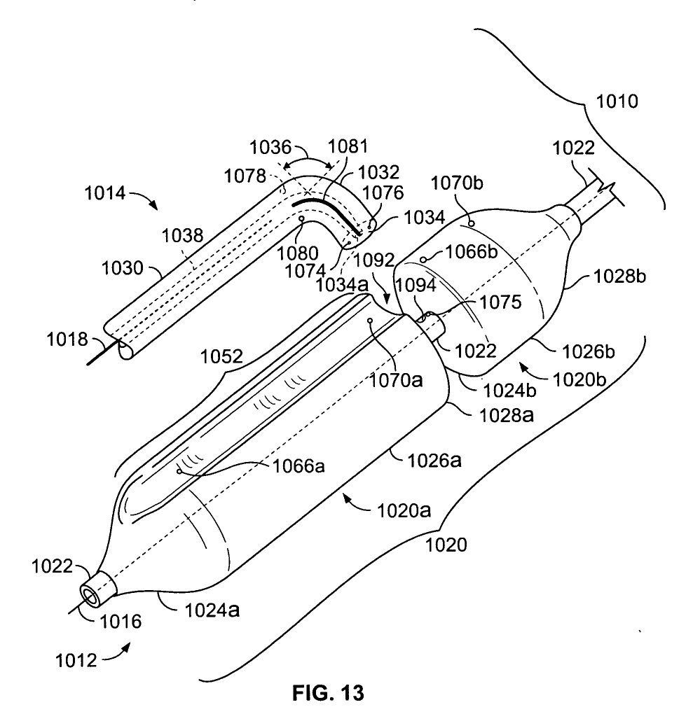

FIGS. 13-20 depict a second exemplary embodiment of the present invention. Elements illustrated in FIGS. 13-20, which correspond, either identically or substantially, to the elements described above with respect to the embodiment of FIGS. 1-12, have been designated by corresponding reference numerals increased by one thousand. Unless otherwise stated or illustrated, the embodiment of FIGS. 13-20 is constructed and operates in the same basic manner as the embodiment of FIGS. 1-12.

With reference to FIG. 13, there is shown a system 1010 for treating patients suffering from CTO occurring in the lower extremities, in accordance with a second exemplary embodiment of the present invention. The system 1010, which may be used in conjunction with the inventive methods described hereinbelow, includes a capture catheter 1012, an angled catheter 1014 and guide-wires 1016, 1018. More particularly, each of the guide-wires 1016, 1018 has a conventional construction. For purposes of clarity, the angled catheter 1014 is shown in FIG. 13 in a scale somewhat larger than that of the capture catheter 1012.

Referring to 13-14B, the capture catheter 1012 includes a balloon assembly 1020 which has a head balloon 1020a and a tail balloon 1020b. The head balloon 1020a, which is positioned distal from the tail balloon 1020b, has a distal end 1024a, a proximal end 1028a and a generally cylindrical middle portion 1026a. Similarly, the tail balloon 1020b has a distal end 1024b, a proximal end 1028b and a generally cylindrical middle portion 1026b. The tail balloon 1020b is spaced axially from the head balloon 1020a such that an annular space 1092 is formed between the proximal end 1028a of the head balloon 1020a and the distal end 1024b of the tail balloon 1020b for purposes to be discussed hereinbelow.

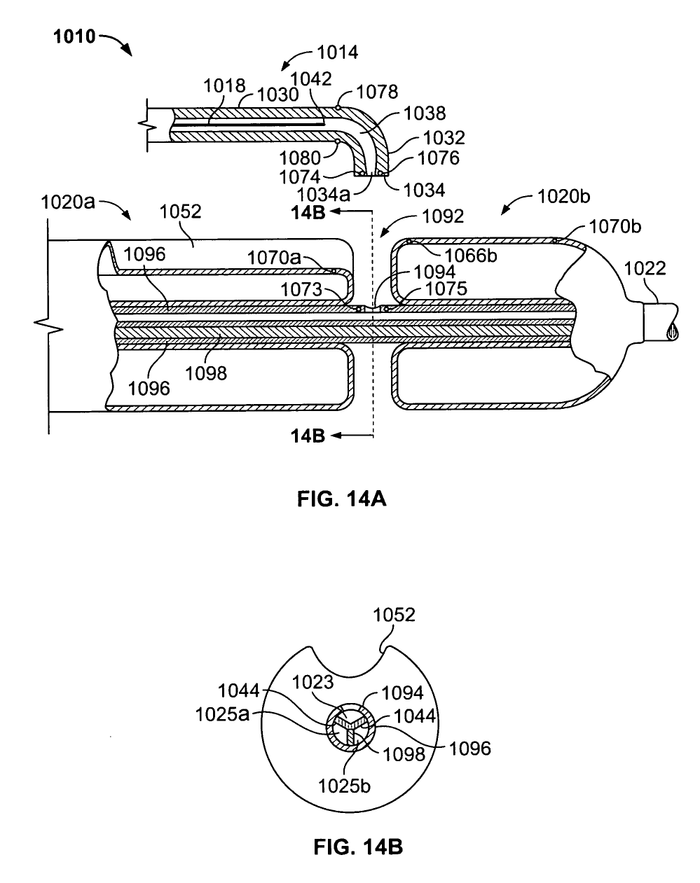

Still referring to FIGS. 13-14B, the capture catheter 1012 includes an elongate tubular body 1022 (i.e., a carrier) having an orifice 1094 which is positioned between the head and tail balloons 1020a, 1020b and which therefore is open to the space 1092. The elongate tubular body 1022 is flexible and is made in a conventional manner. More particularly, the elongate tubular body 1022 is adapted to traverse over the guide-wire 1016 so as to deliver/retrieve the head and tail balloons 1020a, 1020b to/from a CTO, and to permit the head and tail balloons 1020a, 1020b to be remotely inflated and deflated. For such purposes, the elongate tubular body 1022 is provided with a tubular wall 1096 (see FIG. 14B) and a lumen 1023 which extends axially through the tubular wall 1096 and which is sized and shaped so as to permit passage of the guide-wire 1016 or the guide-wire 1018 therethrough. Lumens or passages 1025a, 1025b (see FIG. 14B) also extend axially through the tubular wall 1096. Each of the lumens 1025a, 1025b is separated from the lumen 1023 by a wall 1044 (see FIG. 14B). In addition, a wall 1098 separates the lumen 1025a from the lumen 1025b. The lumen 1025a is in fluid communication with the head balloon 1020a via one or more holes (not shown) formed in the tubular wall 1096, while the lumen 1025b is in fluid communication with the tail balloon 1020b via one or more holes (not shown) formed in the tubular wall 1096. The lumens 1025a, 1025b are sized and shaped so as to permit passage of pressurized fluid therethrough for selectively and independently inflating and deflating the head and tail balloons 1020a, 1020b, respectively. Alternatively, the lumens 1025a, 1025b can be combined as a single lumen and/or be in fluid communication with each other such that the head and tail balloons 1020a, 1020b can be inflated and deflated simultaneously.

With reference to FIGS. 13 and 14A, the angled catheter 1014 is of a construction similar in many respects to that of a straight catheter, but with some differences. For example, the angled catheter 1014 includes an elongate portion 1030 and an end portion 1032 (the later terminating at a tip 1034 with an opening 1034a), but the end portion 1032 is disposed at an angle 1036 relative to the elongate portion 1030, rather than being axially aligned therewith. The length of the end portion 1032 of the angled catheter 1014 can preferably be about 1.3 mm, but may be varied depending on need. The angle 1036 of the angled catheter 1014 is typically about 90 degrees, but may also be varied depending on need. The angled catheter 1014 also includes a lumen 1038 which is sized and shaped so as to accommodate the guide-wire 1018. More particularly, the lumen 1038 extends through the elongate portion 1030 and the end portion 1032 and terminates at the opening 1034a of the tip 1034. Unlike the end portion 32 of the embodiment shown in FIGS. 1-12, the end portion 1032 is preferably not tapered and therefore has a diameter similar to the diameter of the elongate portion 1030. Depending upon specific applications, the end portion 1032 can be tapered like the end portion 32 shown in FIGS. 1 and 2.

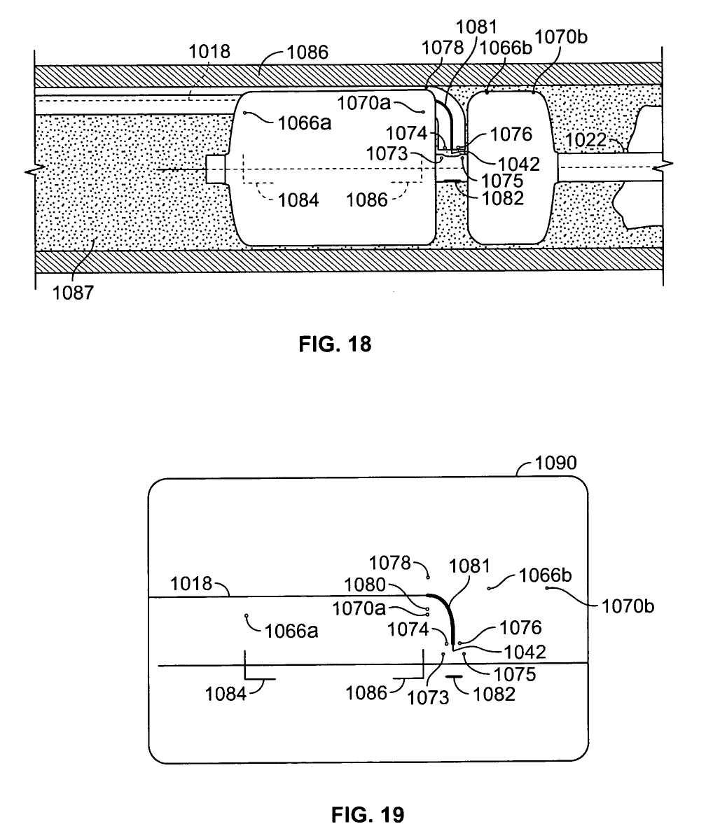

Still referring to FIGS. 13 and 14A, the capture catheter 1012 includes certain structures and other features for enabling a competent practitioner to position the angled catheter 1014 in a proper orientation within a vascular body (e.g., a blood vessel) relative to the capture catheter 1012 and to pass or transfer an end 1042 of the guide-wire 1018 from the angled catheter 1014 into the lumen 1023 of the capture catheter 1012. More particularly, the head balloon 1020a is configured such that when it is inflated, it defines an elongated trough 1052 which is open to the exterior of the head balloon 1020a and which extends axially along a side thereof from the proximal end 1028a and terminates at or adjacent the distal end 1024a. When the head balloon 1020a is inflated, the trough 1052 is sized and shaped so as to receive a section of the elongate portion 1030 of the angled catheter 1014 and is preferably provided with a depth slightly smaller than the diameter of the angled catheter 1014 (which can preferably be about 1.3 mm, but may be varied according to need). The head and tail balloons 1020a, 1020b are also spaced from each other by a predetermined distance (e.g., 1.5 mm to 2.0 mm) so as to form the annular space 1092 when the head and tail balloons 1020a, 1020b become inflated. When the head and tail balloons 1020a, 1020b are inflated, their outside diameter can preferably be about 6 mm (other sizes are also possible). When positioned in the annular space 1092, the tip 1034 of the angled catheter 14 is retained between the head and tail balloons 1020a, 1020b, thereby facilitating the alignment of the tip 1034 with the orifice 1094 of the capture catheter 1012 (see, e.g., FIG. 18). The tip 1034 of the angled catheter 1014 has a predetermined size such that when it is aligned in the aforesaid position, it is spaced from the orifice 1094 of the capture catheter 1012 by a relatively small distance (e.g., 0.4 mm).

With reference to FIGS. 13, 14A and 15, the capture catheter 1012 and the angled catheter 1014 are each equipped with small, discrete portions of radio-opaque material that are positioned or embedded at selected locations in each such component. More particularly, spherical shaped radio-opaque markers 1066a, 1070a (see FIG. 13) are located at the bottom of the trough 1052 adjacent the distal and proximal ends 1024a, 1028a, respectively, of the head balloon 1020a. Similarly, radio-opaque markers 1066b, 1070b are positioned on the middle portion 1026b of the tail balloon 1020b adjacent to the distal and proximal ends 1024b, 1028b, respectively.

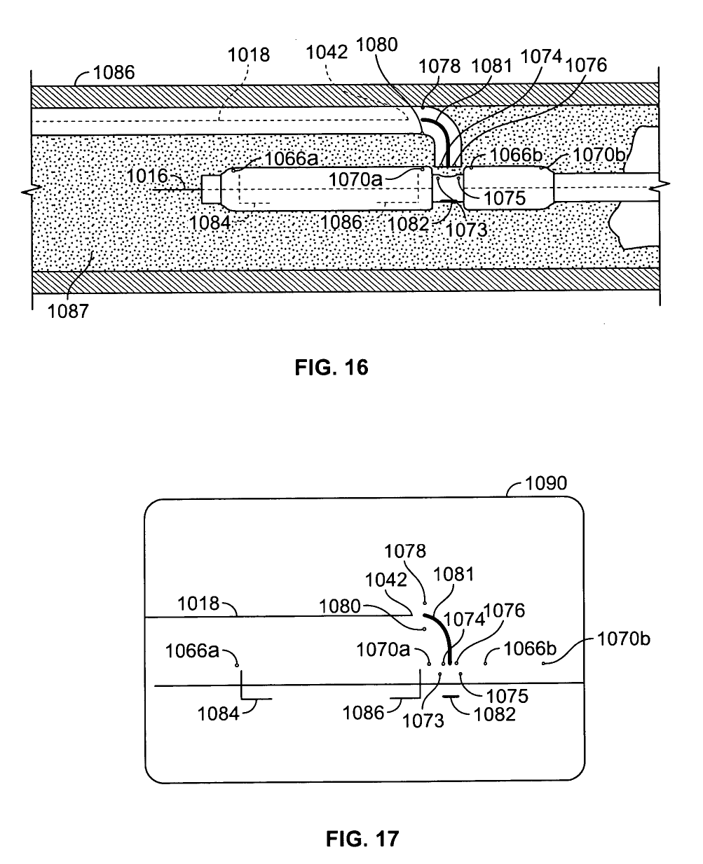

Referring to FIGS. 13 and 16, the angled catheter 1014 includes radio-opaque markers 1074, 1076 disposed on the tip 1034 on opposite sides of the opening 1034a, as well as radio-opaque markers 1078, 1080 disposed on vertically opposing sides of the elongate portion 1030 adjacent to the angle 1036. The angled catheter 1014 is also provided with a curved radio-opaque marker 1081 placed on an outer surface of the angled catheter 1014. The radio-opaque marker 1081 extends from the angle 1036 and terminates at the tip 1034. The radio-opaque marker 1081 is also positioned such that it overlies the lumen 1038, and its curvature of the radio-opaque marker 1081 is similar to that of the lumen 1038.

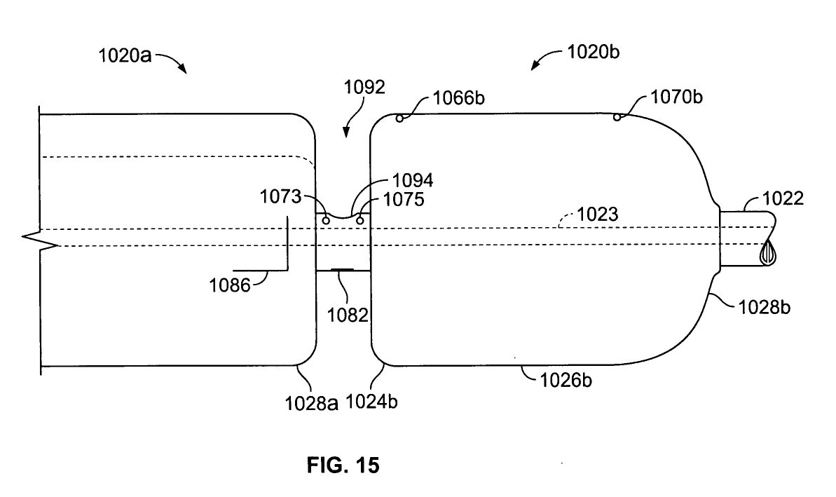

FIG. 15 shows that a lower portion 1082 of the elongated tubular body 1022 of the capture catheter 1012 is coated with and/or constructed of a radio-opaque material. In addition, the elongate tubular body 1022 is provided with a plurality of radio-opaque markers 1073, 1075, disposed on opposite sides of the orifice 1094 of the capture catheter 1012, so as to facilitate alignment of the tip 1034 of the angled catheter 1014 with the orifice 1094 of the capture catheter 1012. Radio-opaque markers 1084, 1086 (see also FIG. 16), each of which has an L-shape, are also positioned on a side surface of the elongate body 1022 so as to facilitate alignment of the orifice 1094 of the elongated body 1022 with the tip 1034 of the angled catheter 1014.

With reference to FIGS. 16-20, the system 1010 illustrated in FIGS. 13-15 can be used to facilitate proper axial positioning of the guide-wire 1018 within an occluded region (i.e., a treatment site) of a blood vessel. As described above, good axial positioning of a guide-wire improves the chances that a later-placed treatment balloon (not shown) will, when inflated, compress the blockage against the vessel wall in approximately equal amounts. What follows below is a discussion of a method of using the system 1010 so as to properly position the guide-wire 1018 through an occluded region. Unless otherwise stated or illustrated, the method utilized in connection with the system 1010 is basically identical to the method utilized in conjunction with the embodiment shown in FIGS. 1-12.

Referring primarily to FIGS. 16 and 17, the guide-wire 1016, placed percutaneously, is advanced downstream through a vascular body or structure 1086 (e.g., an arterial lumen) such that it is placed within a treatment site 1087 (referred to hereinafter as “the CTO region”) where a CTO is present. If the CTO region 1087 is present in a lower extremity of a patient, the guide-wire 1016 is preferably introduced into the vascular structure 1086 through a puncture made at a region at or near the patient's thigh. Once the guide-wire 1016 is properly positioned, the capture catheter 1012 is then advanced along the guide wire 1016 such that it is positioned in the CTO region 1087 (see FIG. 16).

The guide-wire 1018 is also introduced into the vascular structure 1086 from an area distal from the CTO region 1087 and is advanced upstream to a point just distal to the CTO region 1087. The guide-wire 1018 can be introduced into the vascular structure 1086 in any conventional manner. For instance, if the CTO region 1087 is in a lower extremity of the patient, the guide-wire 1018 can be inserted into the vascular structure 1086 through an incision made near or at the patient's ankle or foot with the use of a method known in the art. If, however, the pulse in the vascular structure 1086 is non-palpable (i.e., not easily detectable by touch) or difficult to detect due to, for instance, the fact that blood flow is restricted by the CTO condition, a Doppler-guided needle (e.g., Doppler-guided needles sold by Escalon Vascular Access Inc. under the trademark PD Access Percutaneous Doppler Access System) can be used. More particularly, using a Doppler-guided needle, a surgeon can locate a hard-to-find vascular structure with the aid of an ultrasonic detector and then insert the needle into the vascular structure. The guide-wire 1018 can then be inserted into the vascular structure through the needle.

After advancing the guide-wire 1018 through the vascular structure 1086 to a location just distal to the CTO region 1087, a conventional straight catheter (not shown), similar to the catheter 88 depicted in FIG. 4, is moved upstream through the CTO region 1087 along the plane of dissection. The straight catheter facilitates the passage of the guide-wire 1018 through the plane of dissection, as it crosses the CTO region 1087. The guide-wire 1018 passes through the CTO region 1087 with the aid of the straight catheter until it overlaps with the capture catheter 1012 within the CTO region 1087. The straight catheter is then removed and replaced with the angled catheter 1014. More particularly, the straight catheter is withdrawn from the CTO region 1087 by pulling along the guide-wire 1018 and exiting through the skin of the patient at its original point of entry, leaving just the guide-wire 1018 in place within the vascular structure 1086. The angled catheter 1014 is then introduced to the patient via the point of entry used by the straight catheter, and advanced over the guide-wire 1018. The end portion 1032 of the angled catheter 1014 is preferably made from an elastic material such that the end portion 1032 can be oriented from its normal, angled orientation (as shown in FIG. 16) to a substantially linear orientation relative to the elongate portion 1030. As a result, the end portion 1032 can be passed through the CTO region 1087 in its linear orientation so as to facilitate passage therethrough.

After positioning the capture catheter 1012 and the end portion 1032 of the angled catheter 1014 at the CTO region 1087, the axial and angular orientation of the capture catheter 1012 and/or the angled catheter 1014 is adjusted for proper alignment. Referring to FIGS. 16 and 17, in order to properly position the capture catheter 1012 relative to the angled catheter 1014, a practitioner can use a radioscope display 1090 (see FIG. 17) to remotely view the guide-wires 1016, 1018, as well as the radio-opaque markers 1066a, 1070a, 1066b, 1070b, 1073, 1075, 1082, 1084, 1086, (see also FIGS. 13-15) of the capture catheter 112 and the radio-opaque markers 1074, 1076, 1078, 1080, 1081 (see also FIG. 14A) of the angled catheter 1014. With the aid of the radioscope display 1090, the capture catheter 1012 and/or the angled catheter 1014 can be moved axially and/or rotated relative to each other and/or around their respective guide-wires as necessary. For instance, the images of the radio-opaque markers 1066a, 1070a, 1066b, 1070b, 1073, 1075, 1082, 1084, 1086, 1074, 1076, 1078, 1080, 1081 appearing on the radioscope display 1090 can be used for adjusting the angular orientation of the capture catheter 1012. For instance, the capture catheter 1012 can be rotated until the vertical portions of the “L” shaped radio-opaque markers 1084, 1086 appear at their maximum on the radioscope display 1090. The markers 1084, 1086 are arranged on the side surface of the elongate body 22 such that, if the trough 1052 of the head balloon 1020a is not in substantial angular alignment with the angled catheter 1014, one or both of the markers 1084, 1086 will not be visible on the radioscope display 1090, or their vertical portions will appear short on the radioscope display 1090. In order to adjust the angular orientation of the head balloon 1020a, the capture catheter 1012 is rotated until the marker 1084, 1086 become visible on the radioscope display 1090 and/or until the respective vertical portions of the markers 1084, 1086 appear with their maximum lengths on the radioscope display 1090.

The angular orientation of the angled catheter 1014 can also be adjusted in a similar manner by viewing the radio-opaque markers 1074, 1076, the radio-opaque makers 1078, 1080 and/or the radio-opaque marker 1081. For instance, the angular orientation of the angled catheter 1014 can be adjusted by rotating the angled catheter 1014 until the radio-opaque marker 1081 becomes visible on the radioscope display 1090 and/or until the vertical portion of the radio-opaque marker 1081 appears at its maximum on the radioscope display 1090.

One or more of the images appearing on the radioscope display 1090 of the radio-opaque markers 1066a, 1070a, 1066b, 1070b, 1073, 1074, 1075, 1076, 1078, 1080, 1081 can also be used to verify whether the orifice 1094 of the capture catheter 1012 is axially and/or angularly aligned with the opening 1034a of the end portion 1034 of the angled catheter 1014. For instance, if the radio-opaque markers 1074, 1076 of the angled catheter 1014 appear on the radioscope display 1090 axially between, and immediately above, the radio-opaque markers 1073, 1075 of the capture catheter 1012, such positioning indicates that the end portion 1032 is axially aligned with the orifice 1094. If such alignment is not indicated by the radioscope display 1090, the angled catheter 1014 and/or the capture catheter 1012 can be moved axially to achieve proper alignment. Moreover, the angled catheter 1014 can be rotated, and the images of the radio-opaque markers 1074, 1076, 1078, 1080, 1081 can be monitored on the radioscope display 1090 so as to verify that the opening 1034a of the end portion 1032 of the angled catheter 1014 is angularly aligned with the orifice 1094 of the capture catheter 1012.

By the end of the alignment procedure discussed above, the opening 1034a of the end portion 1032 of the angled catheter 1014 should be positioned directly above, and facing directly toward, the orifice 1094 of the capture catheter 1012 (see FIG. 16). In this alignment, when the head and tail balloons 1020a, 1020b are inflated, the end portion 1032 of the angled catheter 1014 can be positioned and retained in the annular space 1092 between the head and tail balloons 1020a, 1020b in order to maintain proper alignment between the angled catheter 1014 and the capture catheter 1012 during subsequent procedures which will be discussed in greater detail hereinbelow.

Referring now to FIG. 18, once proper alignment between the opening 1034a of the end portion 1032 of the angled catheter 1014 and the orifice 1094 of the capture catheter 1012 has been achieved, the head and tail balloons 1020a, 1020b are inflated either sequentially or simultaneously. As the head and tail balloons 1020a, 1020b become inflated, they expand radially outwardly and form the annular space 1092 therebetween. As a result, the end portion 1032 of the angled catheter 1014 is placed in the annular space 1092. The annular space 1092 is provided with a predetermined axial width such that the proximal end 1028a of the head balloon 1020a and the distal end 1024b of the tail balloon 1020b snugly engage the end portion 1032 of the angled catheter 1014 so as to retain same therein and to thereby inhibit the angled catheter 1014 from moving axially relative to the orifice 1094 of the capture catheter 1012. Moreover, as the head balloon 1020a expands radially outwardly, it causes the trough 1052 to form and positions a portion of the elongate portion 1030 of the angled catheter 1014 in the trough 1052. As a result, the elongate portion 1030 of the angled catheter 1014 is captured in the trough 1052 (due to the fact that the elongate portion 1030 is retained between the trough 1052 and the vascular wall), thereby inhibiting angular and/or axial movement of the angled catheter 1014. The practitioner can use the radioscope display 1090 to remotely view (see FIG. 19) the guide-wires 1016, 1018, and the radio-opaque markers 1066a, 1070a, 1066b, 1070b, 1073, 1075, 1082, 1084, 1086 of the capture catheter 1012 and the radio-opaque markers 1074, 1076, 1078, 1080, 1081 of the angled catheter 1014, so as to confirm proper engagement between the angled catheter 1014 and the capture catheter 1012.

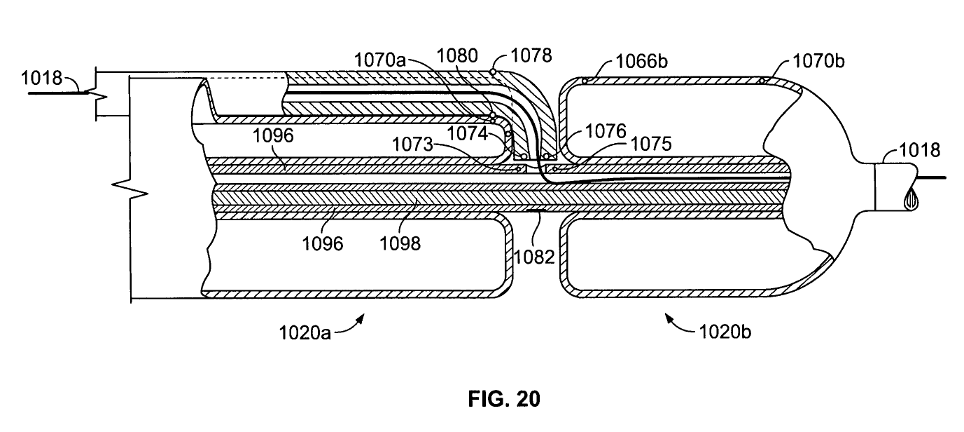

The nature of the engagement between the angled catheter 1014 and the capture catheter 1012 is illustrated in detail in FIGS. 18 and 20. More particularly, the tip 1034 of the angled catheter 1014 is positioned directly over and pointing towards the orifice 1094 of the elongated tubular body 1022 of the capture catheter 1012. This is remotely confirmed by the practitioner via images appearing on the radioscope display 1090 as shown in FIG. 19. The guide-wire 1018 can now be advanced into the capture catheter 1012, and into the lumen 1023 and out of the vascular structure 1086 of the patient. This process will now be described below with reference to FIG. 20.

The guide-wire 1016 preferably remains within the capture catheter 1012 until the head and tail balloons 1020a, 1020b are fully inflated and is thereafter removed from the lumen 1023 of the capture catheter 1012. After removing the guide-wire 1016 from the capture catheter 1012, the end 1042 of the guide-wire 1018 is passed through the opening 1034a of the angled catheter 1014 and then fed into the orifice 1094 of the elongated tubular body 1022 of the capture catheter 1012. The guide-wire 1018 is thereafter advanced upstream through the lumen 1023 of the elongate tubular body 1022 of the capture catheter 1012, and out of the vascular structure 1086 of the patient so that the end 1042 of the guide-wire 1018 is outside of the patient's body and can be grasped or otherwise manipulated by the practitioner. At this point, the practitioner has a much greater ability to manipulate the axial position of the guide-wire 1018 (from two ends) than was the case when one end of a guide-wire was merely positioned at the upstream end of a CTO region. The capture catheter 1012 can now be removed from the vascular structure 1086 after the head and tail balloons 1020a, 1020b have been deflated.

After the guide-wire 1018 is positioned properly through the CTO region 1087, a conventional treatment balloon (not shown) can be tracked over the guide-wire 1018 from the upstream or downstream entry point in the body (not shown). After positioning the treatment balloon in a desired location within the CTO region 1087, the treatment balloon is inflated so as to push the CTO against the walls of the vascular structure 1086, thus enlarging the opening made by the guide-wire 1018.

It should be noted that the system 1010 and the method or methods of use associated therewith can have numerous modifications and variations. For instance, the system 1010 may be used without the tail balloon 1020b or the head balloon 1020a. In addition, one or some of the radio-opaque markers 1066a, 1070a, 1066b, 1070b, 1073, 1075, 1082, 1084, 1086, 1074, 1076, 1078, 1080, 1081 can be eliminated and/or replaced with one or more additional radio-opaque markers. By way of example, L-shaped radio-opaque markers similar to the L-shaped radio-opaque markers 1084, 1086 can be provided on the angled catheter 1014. Moreover, the radio-opaque markers can be replaced with other known mechanisms. Further, the capture catheter 1012 can be advanced to the CTO region 1087 from an entry point which is located downstream from the CTO region 1087.

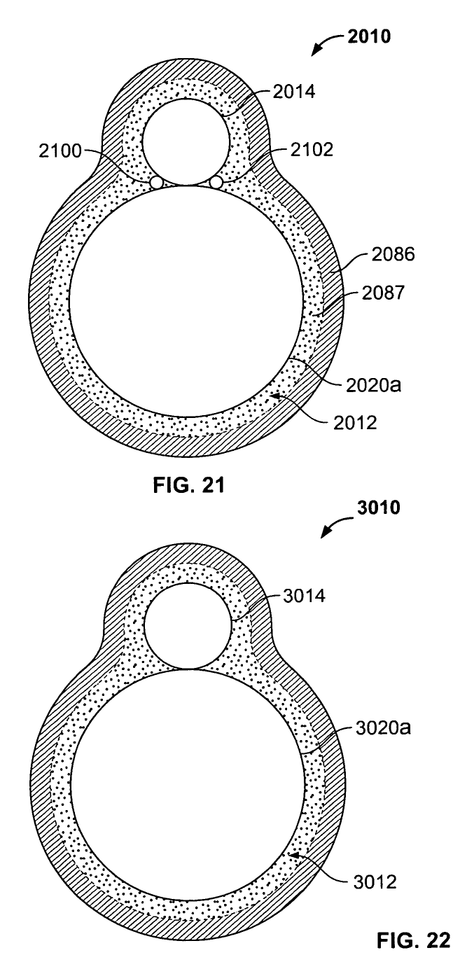

FIG. 21 depicts a third exemplary embodiment of the present invention. Elements illustrated in FIG. 21, which correspond, either identically or substantially, to the elements described above with respect to the embodiment of FIGS. 13-20, have been designated by corresponding reference numerals increased by one thousand. Unless otherwise stated or illustrated, the embodiment of FIG. 21 is constructed and operates in the same basic manner as the embodiment of FIGS. 13-20.

FIG. 21 schematically illustrates a system 2010 constructed in accordance with the third embodiment of the present invention. The system 2010 includes an angled catheter 2014 which is identical to the angled catheter 1014 of the embodiment shown in FIGS. 13-20 (hereinafter “the second embodiment”). The system 2010 also includes a capture catheter 2012 having a head balloon 2020a and a tail balloon (not shown). The capture catheter 2012 is identical to the capture catheter 1012 of the second embodiment, except as discussed below. The head balloon 2020a is provided with a pair of guide rods 2100, 2102 which replace the trough 1052 of the head balloon 1020a of the second embodiment. As a result, the head balloon 2020a has a substantially circular cross-section along its entire axial length. The guide rods 2100, 2102, which can be made from any conventional material (e.g., any metallic or alloy material known in the art), are mounted on an outer surface of the head balloon 2020a and extends in an axial direction along the substantially entire length of the head balloon 2020a. The guide rods 2100, 2102 are spaced laterally from one another and cooperate so as to perform basically the same function or functions as the trough 1052 of the second embodiment (e.g., engaging the angled catheter 2014 when the head balloon 2020a is inflated so as to inhibit the angled catheter 2014 from moving laterally and to thereby maintain alignment of the angled catheter 2014 with respect to the capture catheter 2012). The system 2010 is placed within a CTO region 2087 of a vascular vessel 2086 in a manner basically identical to that of the second embodiment.

It should be noted that the guide rods 2100, 2102 can be replaced with any mechanism that can perform the same function or functions. For example, the guide rods 2100, 2102 can be replaced with wing-like inflatable membranes extending longitudinally along the head balloon 2020a. Such inflatable members, when inflated, can form a channel which performs the same basic function as the guide rods 2100, 2102 (e.g., receiving and stabilizing the angled catheter 2014). If such inflatable members are utilized, the diameter of the head balloon 2020a may need to be reduced to compensate for the increase in diameter as a result of the inflatable members. The rods 2100, 2102 can also be replaced with foldable blades mounted to the head balloon 2020a.

FIG. 22 depicts a fourth exemplary embodiment of the present invention. Elements illustrated in FIG. 22, which correspond, either identically or substantially, to the elements described above with respect to the embodiment of FIG. 21, have been designated by corresponding reference numerals increased by one thousand. Unless otherwise stated or illustrated, the embodiment of FIG. 22 is constructed and operates in the same basic manner as the embodiment of FIG. 21.

Referring to FIG. 22, there is shown a system 3010 constructed in accordance with the fourth embodiment of the present invention. The system 3010 includes a capture catheter 3012 having a head balloon 3020a. The head balloon 3020a is identical to the head balloon 2020a of the embodiment of FIG. 21 (referred to hereinafter as “the third embodiment”), except that the head balloon 2020a is not equipped with any mechanism similar to the guide rods 2100, 2102 of the third embodiment. Because the head balloon 3020a is not provided with any such mechanism, a practitioner may opt to inflate the head balloon 3020a, as well as its associated tail balloon (not shown), at a slow rate such that the head balloon 3020a can slowly engage an associated angled catheter 3014 so as to prevent misalignment of the capture catheter 3012 with respect to the angled catheter 3014.

It should be noted that the present invention can have numerous modifications and variations. For instance, the size, shape and construction of the balloons associated with the embodiments described above may vary while still performing the same functions. Moreover, as mentioned above, the capture catheter can be provided with only one balloon, which may or may not include a trough, guide rods or other guiding mechanisms, for engaging the angled catheter. The angled tip of the angled catheter can also be eliminated (i.e., the angled catheter can be a straight catheter).

It will be understood that the embodiments described herein are merely exemplary and that a person skilled in the art may make many variations and modifications, including those discussed above, without departing from the spirit and scope of the invention. All such variations and modifications are intended to be included within the scope of the invention as defined in the appended claims.

What is claimed is:

1. A catheter system for positioning a guide wire through a treatment site within a vascular body, comprising a first catheter having a first lumen extending therethrough; and a second catheter having a second lumen extending therethrough, said second catheter including engaging means for engaging at least a portion of said first catheter such that a guide wire can be fed from said first lumen of said first catheter to said second lumen of said second catheter.

2. The catheter system of claim 1, wherein said engaging means includes at least one inflatable balloon which is engageable with said first catheter when said at least one balloon is inflated.

3. The catheter system of claim 2, wherein said at least one balloon includes a first inflatable balloon and a second inflatable balloon spaced from one another axially so as to form a space therebetween.

4. The catheter system of claim 3, wherein said second catheter includes a carrier, said first and second balloons being attached to said carrier, said second lumen extending axially through said carrier.

5. The system of claim 4, wherein said carrier includes an orifice formed on a side wall thereof between said first and second balloons and communicating with said second lumen and said space so as to permit passage of a guide wire fed from said first lumen of said first catheter into said second lumen of said second catheter through said space and orifice.

6. The system of claim 3, wherein said first catheter include an angled tip.

7. The system of claim 6, wherein said space is sized and shaped so as to receive said angled tip of said first catheter when said first and second balloons are inflated.

8. The system of claim 7, wherein said first and second balloons are sized and shaped so as to engage said angled tip when said first and second balloons are inflated.

9. The system of claim 8, wherein said first catheter includes an elongated portion.

10. The system of claim 9, wherein said angled tip is oriented at an angle relative to said elongate portion.

11. The system of claim 10, wherein said first lumen extends axially through said elongate portion, said first lumen extending through said angled tip.

12. The system of claim 2, wherein said at least one balloon is configured such that it forms a trough along a side thereof when it is inflated.

13. The system of claim 12, wherein said trough is sized and shaped so as to receive said at least a portion of said first catheter.

14. The system of claim 2, wherein said at least one balloon includes at least one guide member for engaging said at least a portion of said first catheter.

15. The system of claim 14, wherein said at least one guide member includes first and second guide members attached to said at least one balloon.

16. The system of claim 15, wherein each of said first and second guide members extends axially along said at least one balloon.

17. The system of claim 2, wherein said at least one balloon includes an opening sized and shaped such that a guide wire can be fed from said first lumen of said first catheter into said second lumen of said second catheter through said opening.

18. The system of claim 17, wherein said at least one balloon includes a trough formed adjacent an outer periphery thereof when said balloon is inflated, said trough being sized and shaped so as to receive said at least a portion of said first catheter, said trough communicating with said second lumen through said opening.

19. The system of claim 18, wherein said second catheter includes a carrier, said at least one balloon being attached to said carrier, said opening extending in a generally radial direction and being positioned between said trough and said carrier, said carrier including an orifice so as to permit communication between said opening and said lumen.

20. The system of claim 1, further comprising indicating means for indicating the orientation of said first catheter relative to said second catheter within a vascular body.

21. The system of claim 20, wherein said indicating means includes a plurality of first markers positioned on said first catheter, said first markers being viewable on a remote display.

22. The system of claim 21, wherein said indicating means includes a plurality of second markers provided on said second catheter, said second markers being viewable on a remote display.

23. The system of claim 22, wherein said engaging means includes at least one inflatable balloon, said second markers being provided on said at least one balloon.

24. The system of claim 23, wherein said second catheter includes a carrier, said at least one balloon attached to said carrier, said indicating means includes a plurality of third markers provided on said carrier, said third markers being viewable on a remote display.

25. The system of claim 24, wherein at least one of said third markers has an L-shape.

26. The system of claim 25, wherein at least one of said first, second and third markers is an radio-opaque marker.

27. The system of claim 1, further comprising first indicating means for indicating the axial orientation of said first catheter relative to said second catheter within a vascular body and second indicating means for indicating the angular orientation of said first catheter relative to said second catheter within a vascular body.

28. The system of claim 27, wherein said first indicating means includes a plurality of first markers provided on at least one of said first and second catheters; and wherein said second indicating means includes a plurality of second makers provided on at least one of said first and second catheters, said first and second makers being viewable on a remote display.

29. A method for positioning a catheter guide wire through a treatment site in a vascular body, comprising the steps of:
(a) advancing a first catheter to the treatment site through the vascular body from a downstream side of the treatment site;
(b) advancing a second catheter to the treatment site through the vascular body from an upstream side of the treatment site;
(c) engaging the first catheter with the second catheter within the vascular body;
(d) feeding a guide wire from one of the first and second catheters to the other one of the first and second catheters; and
(e) removing the first and second catheters from the vascular body, leaving the guide wire extending through the treatment site.

30. The method of claim 29, further comprising the step of aligning the first catheter relative to the second catheter.

31. The method of claim 30, wherein said aligning step includes the step of rotating at least one of the first and second catheters so as to angularly align the second catheter relative to the first catheter.

32. The method of claim 31, wherein said aligning step includes the step of axially moving the first catheter relative to the second catheter.

33. The method of claim 29, wherein said engaging step includes the step of inflating at least one inflatable balloon attached to the second catheter such that the at least one balloon engages at least a portion of the first catheter.

34. The method of claim 33, wherein the at least one balloon includes a first inflatable balloon and a second inflatable balloon spaced from one another axially so as to form a space therebetween, the first and second balloons being inflated during the performance of said inflating step.

35. The method of claim 33, wherein the at least one balloon forms a trough along a side thereof when it is inflated, the at least a portion of the first catheter being placed in the trough during the performance of said inflating step.

36. The method of claim 29, wherein said feeding step is performed by feeding the guide wire from said first catheter to said second catheter.

37. The method of claim 29, wherein said removing step is performed after the first catheter is disengaged from the second catheter.
